(19) United States
(12) Patent Application Publication
(10) Pub. No.: US 2012/0171144 A1
(43) Pub. Date: Jul. 5, 2012

(54) METHODS OF TREATING, REDUCING AND INHIBITING THE APPEARANCE OF AGEING IN THE SKIN

(76) Inventors: David James Granville, Port Coquitlam (CA); Rani Priya Gomez Cruz, Vancouver (CA)

(21) Appl. No.: 12/282,157

(22) PCT Filed: Mar. 9, 2007
(86) PCT No.: PCT/CA07/00396

§ 371 Date: Nov. 9, 2009

Related U.S. Application Data
(60) Provisional application No. 60/780,352, filed on Mar. 9, 2006.
(60) Provisional application No. 60/797,352, filed on May 4, 2006.

(51) Int. Cl. A61K 8/64; A61Q 19/08; C12Q 1/37; A61Q 5/00

(52) U.S. Cl. 424/70.1; 514/18.8; 435/23

(57) ABSTRACT

Methods of treating, reducing and inhibiting the appearance of ageing in the skin are provided. Also provided are uses and methods of maintaining a youthful appearance, reducing an appearance of ageing, inhibiting an appearance of ageing, reducing a rate of an appearance of ageing, reducing a skin inelasticity, reducing a rate of increasing skin inelasticity, maintaining a skin elasticity, and increasing the density of hair follicles of a skin of a subject comprising applying a granzyme B inhibitor to the skin of the subject. Also provided are methods of identifying a granzyme B inhibitors and agonists.

### FIELD

This invention relates to the field of skin cosmetics. More particularly, it relates to treating, reducing and inhibiting the appearance of ageing of skin using granzyme B inhibitors.

### BACKGROUND

International patent application, published under WO 03/065987 describes the use of granzyme B inhibitors for treating autoimmune and chronic inflammatory diseases as well as other diseases and disorders.

United States patent application, published under 2003/0148511 describes the use of granzyme B inhibitors for enhancing host immunity to a virus and/or cancer as well as methods for enhancing the cytotoxic T-cell (CTL) mediated immune responses.

Buzza et al., The Journal of Biological Chemistry, Volume 280, No. 25, pages 23549-23558 (2005) describes that granzyme B possesses a potent extracellular matrix remodeling activity. Buzza et al. also describes that both native and recombinant granzyme B cause detachment of immortalized and transformed cell lines, primarily endothelial cells and chondrocytes. Buzza et al. also describes that granzyme B cleaves three proteins involved in extracellular matrix structure and function: vitronectin, fibronectin, and laminin.

The skin is one of the largest organs in the body and its condition and appearance are determined, in part, by the amount and the state of elastin that is contained in the skin. Elastin is a matrix protein and is comprised of tropoelastin monomers, cross-linked and organized into larger tertiary structures. Elastin confers elasticity, preventing or inhibiting dynamic tissue creep by stretching under load and enabling the tissue to recoil to the original configurations after the load is released. Loss of skin tone or elasticity, stiffening of joints and loss of flexibility are associated with elastin degradation and alteration in connective tissue structure.

Various compositions and methods for manipulating the quality of skin are available. For example, international patent application, published under number WO 2004/100889, describes anti-ageing agents, including 3,3′-thiodipropionic acid or derivatives for improvement of the aesthetic appearance of skin. One method for manipulating the quality of the skin is cosmetic surgery. Other methods involve application of caustic compositions alone or in combination with physical sloughing of the outer layers of the skin, such as ‘dermabrasion’ and ‘chemical peel’ processes. Many compositions for manipulating the quality of skin are not pharmaceuticals for the treatment of a disease, but rather they alter a normal state of the skin such as sagging or wrinkling. These normal skin states are often associated with elastin, an extracellular protein, degradation.

### SUMMARY

This invention is based, in part, on the observation that granzyme B colocalizes with elastin in a skin and in the proximity of elastin in the skin. This invention is also based in part on the observation that granzyme B cleaves elastin, in addition to other extracellular matrix proteins, in the interstitial space surrounding cells and plasma.

In one aspect of the present invention there is provided a method of maintaining a youthful appearance of a skin of a subject comprising applying a granzyme B inhibitor to the skin of the subject.

In another aspect of the present invention there is provided a method of reducing an appearance of ageing of a skin of a subject comprising applying a granzyme B inhibitor to the skin of the subject.

In another aspect of the present invention there is provided a method of inhibiting an appearance of ageing of a skin of a subject comprising applying a granzyme B inhibitor to the skin of the subject.

In another aspect of the present invention there is provided a method of reducing a rate of an appearance of ageing of a skin of a subject comprising applying a granzyme B inhibitor to the skin of the subject.

In another aspect of the present invention there is provided a method of reducing a skin inelasticity in a subject comprising applying a granzyme B inhibitor to a skin of the subject.

In another aspect of the present invention there is provided a method of reducing a rate of increasing skin inelasticity in a subject comprising applying a granzyme B inhibitor to a skin of the subject.

In another aspect of the present invention there is provided a method of maintaining a skin elasticity in a subject comprising applying a granzyme B inhibitor to a skin of the subject.

In another aspect of the present invention there is provided a method of increasing the density of hair follicles in a subject comprising applying a granzyme B inhibitor to a skin of the subject.

In another aspect of the present invention there is provided a use of a granzyme B inhibitor for maintaining a youthful appearance of a skin of a subject.

In another aspect of the present invention there is provided a use of a granzyme B inhibitor for reducing an appearance of ageing of a skin of a subject.

In another aspect of the present invention there is provided a use of a granzyme B inhibitor for inhibiting an appearance of ageing of a skin of a subject.

In another aspect of the present invention there is provided a use of a granzyme B inhibitor for reducing a rate of appearance of ageing of a skin of a subject.

In another aspect of the present invention there is provided a use of a granzyme B inhibitor for reducing a skin inelasticity of a subject.

In another aspect of the present invention there is provided a use of a granzyme B inhibitor for reducing a rate of increasing skin inelasticity of a subject.

In another aspect of the present invention there is provided a use of a granzyme B inhibitor for maintaining a skin elasticity of a skin of a subject.

In another aspect of the present invention there is provided a use of a granzyme B inhibitor for increasing a density of hair follicles of a skin of a subject.

In another aspect of the present invention there is provided a granzyme B inhibitor for use in maintaining a youthful appearance of a skin of a subject.

In another aspect of the present invention there is provided a granzyme B inhibitor for use in reducing an appearance of ageing of a skin of a subject.

In another aspect of the present invention there is provided a granzyme B inhibitor for use in inhibiting an appearance of ageing of a skin of a subject.

In another aspect of the present invention there is provided a granzyme B inhibitor for use in reducing a rate of appearance of ageing of a skin of a subject.

In another aspect of the present invention there is provided a granzyme B inhibitor for use in reducing a skin inelasticity of a skin of a subject.

In another aspect of the present invention there is provided a granzyme B inhibitor for use in reducing a rate of increasing skin inelasticity of a skin of a subject.

In another aspect of the present invention there is provided a granzyme B inhibitor for use in maintaining a skin elasticity of a skin of a subject.

In another aspect of the present invention there is provided a granzyme B inhibitor for increasing the density of hair follicles of a skin of a subject.

In another aspect of the present invention there is provided methods and uses described herein wherein an extracellular protein content of the skin is maintained or increased.

In another aspect of the present invention there is provided methods and uses described herein wherein a skin elastin content of the skin is maintained or increased.

In another aspect of the present invention there is provided methods and uses described herein wherein a skin elasticity of the skin is maintained or increased.

In another aspect of the present invention there is provided methods and uses described herein wherein a skin fragility of the skin is maintained or reduced.

In another aspect of the present invention there is provided methods and uses described herein wherein a skin firmness of the skin is maintained or increased.

In another aspect of the present invention there is provided methods and uses described herein wherein a skin flakiness of the skin is maintained or reduced.

In another aspect of the present invention there is provided methods and uses described herein wherein a skin dryness of the skin is maintained or reduced.

In another aspect of the present invention there is provided methods and uses described herein wherein a pore size of the skin is maintained or reduced.

In another aspect of the present invention there is provided methods and uses described herein wherein a skin thickness is maintained or increased.

In another aspect of the present invention there is provided methods and uses described herein wherein a rate of skin cell turnover is maintained or increased.

In another aspect of the present invention there is provided methods and uses described herein wherein an appearance of wrinkles in the skin of the subject is maintained or reduced.

In another aspect of the present invention there is provided methods and uses described herein wherein a depth of wrinkles is maintained or reduced.

In another aspect of the present invention there is provided methods and uses described herein wherein an appearance of fine lines in the skin of the subject is maintained or reduced.

In another aspect of the present invention there is provided methods and uses described herein wherein an appearance of skin discolouration of the skin of the subject is maintained or reduced.

In another aspect of the present invention there is provided methods and uses described herein wherein a rate of decreasing extracellular protein content of the skin is reduced.

In another aspect of the present invention there is provided methods and uses described herein wherein a rate of decreasing skin elastin content of the skin is reduced.

In another aspect of the present invention there is provided methods and uses described herein wherein a rate of decreasing skin elasticity of the skin is reduced.

In another aspect of the present invention there is provided methods and uses described herein wherein a rate of increasing skin fragility of the skin is reduced.

In another aspect of the present invention there is provided methods and uses described herein wherein a rate of decreasing skin firmness of the skin is reduced.

In another aspect of the present invention there is provided methods and uses described herein wherein a rate of increasing skin flakiness of the skin is reduced.

In another aspect of the present invention there is provided methods and uses described herein wherein a rate of increasing skin dryness of the skin is reduced.

In another aspect of the present invention there is provided methods and uses described herein wherein a rate of pore size enlargement of the skin is reduced.

In another aspect of the present invention there is provided methods and uses described herein wherein a rate of decreasing skin thickness is increased.

In another aspect of the present invention there is provided methods and uses described herein wherein a rate of decreasing skin cell turnover is increased.

In another aspect of the present invention there is provided methods and uses described herein wherein a rate of increasing appearance of wrinkles in the skin of the subject is reduced.

In another aspect of the present invention there is provided methods and uses described herein wherein a rate of increasing depth of wrinkles is reduced.

In another aspect of the present invention there is provided methods and uses described herein wherein a rate of increasing appearance of fine lines in the skin of the subject is reduced.

In another aspect of the present invention there is provided methods and uses described herein wherein a rate of increasing appearance of skin discolouration of the skin of the subject is reduced.

In another aspect of the present invention there is provided methods and uses described herein wherein a grey hair colour is reduced.

In another aspect of the present invention there is provided methods and uses described herein wherein the granzyme B inhibitor is applied topically.

In another aspect of the present invention there is provided methods and uses described herein wherein the granzyme B inhibitor is applied sub-dermally.

In another aspect of the present invention there is provided methods and uses described herein wherein the granzyme B inhibitor is applied to all of the skin of the subject.

In another aspect of the present invention there is provided methods and uses described herein wherein the granzyme B inhibitor is applied to a portion of the skin of the subject.

In another aspect of the present invention there is provided methods and uses described herein wherein the granzyme B inhibitor is applied only to a scalp.

In another aspect of the present invention there is provided methods and uses described herein wherein the subject is a mammal, a domestic pet, a human, or a dog.

In another aspect of the present invention there is provided a method of identifying a granzyme B inhibitor comprising: i) contacting granzyme B with a test compound thereby forming a primed granzyme B; ii) contacting the primed granzyme B with an extracellular skin membrane; and iii) measuring the amount of cleaved extracellular protein, wherein low levels of cleaved extracellular protein indicate the test compound is an inhibitor of granzyme B.

In another aspect of the present invention there is provided a method of identifying a granzyme B inhibitor comprising: i) contacting granzyme B with a test compound thereby forming a primed granzyme B; ii) contacting the primed granzyme B with elastin; and iii) measuring the amount of cleaved elastin, wherein low levels of cleaved elastin indicate the test compound is an inhibitor of granzyme B.

In another aspect of the present invention there is provided a method of identifying a granzyme B agonist comprising: i) contacting granzyme B with a test compound thereby forming a primed granzyme B; ii) contacting the primed granzyme B with an extracellular skin membrane; and iii) measuring the amount of cleaved extracellular protein, wherein high levels of cleaved extracellular protein indicate the test compound is an agonist of granzyme B.

In another aspect of the present invention there is provided a method of identifying a granzyme B agonist comprising: i) contacting granzyme B with a test compound thereby forming a primed granzyme B; ii) contacting the primed granzyme B with elastin; and iii) measuring the amount of cleaved elastin, wherein high levels of cleaved elastin indicate the test compound is an agonist of granzyme B.

### BRIEF DESCRIPTION OF THE DRAWINGS

FIG. 1: a bar graph showing the plasma lipid profiles of C57/B1/6 (solid bar), GrB-KO (white bar), ApoE-KO (hatched bar) or ApoE/GrB-DKO (checked bar) mice on a Western diet. TG average—average triglycerides; TC average—total cholesterol average. N=3 for each group.

FIG. 2: granzyme B degrades elastin in vitro. Granzyme B was incubated with ³H-elastin for 7 days at room temperature. Elastase was incubated with ³H-elastin for 2 hours. Supernatants containing the soluble elastin cleavage fragments were collected and counted. Data is represented as fold increase in radioactivity over the control (elastin only). (n=2)

### DETAILED DESCRIPTION

Granzyme B is a serine protease that is capable of cleaving elastin and other extracellular proteins in the interstitial space surrounding cells and plasma. Inhibiting granzyme B reduces the cleavage of elastin and other extracellular proteins. Reducing the cleavage of elastin and other extracellular proteins improves the condition of, maintains the condition of or reduces a normal deterioration rate of skin.

Granzyme B is an enzyme that can be inhibited. An inhibitor of granzyme B is a substance that will inhibit or slow down the cleavage of extracellular proteins by granzyme B. For example, a compound or composition that prevented granzyme B from cleaving elastin would be a granzyme B inhibitor. In many cases, inhibitors are referred to as antagonists. Conversely, a substance that improves the ability of granzyme B to cleave extracellular proteins is called an agonist. For example, a compound or composition which would increase the rate at which granzyme B cleaves elastin is a granzyme B agonist.

A granzyme B inhibitor may be identified by contacting granzyme B with a test compound in order to form a primed granzyme B. A test compound is a substance, compound or composition that one wishes to identify as an inhibitor of granzyme B or not. A primed granzyme B is a granzyme B enzyme which may or may not have a test compound bound to it and has been in contact or mixed with a test compound. In other words, a primed granzyme B is a granzyme B enzyme under conditions such that by adding an extracellular protein, such as elastin, a test compound may be identified as being an inhibitor or an antagonist of granzyme B or not. Once a primed granzyme B is formed, by contacting it with a predetermined amount of an extracellular protein, such as elastin, it is possible to identify whether or not a particular test compound is a granzyme B inhibitor or antagonist or not by measuring an amount of cleaved extracellular protein that accumulates over a predetermined period of time and comparing the amount of cleaved extracellular protein with a normal amount of cleaved extracellular protein. A normal amount of cleaved extracellular protein can be achieved by adding the same predetermined amount of the extracellular protein to granzyme B, i.e. unprimed granzyme B, and measuring the amount of cleaved extracellular protein that accumulates over the aforementioned predetermined period of time. The predetermined period of time may be any period of time that does not result in cleavage of all of the predetermined amount of the extracellular protein by unprimed granzyme B. A test compound is an inhibitor or antagonist of granzyme B if the amount of the cleaved extracellular protein is less than the normal amount of cleaved extracellular protein. If the amount of cleaved extracellular protein is the same as the normal amount of cleaved extracellular protein, then the test compound is not an inhibitor or antagonist of granzyme B. Alternatively, if the amount of cleaved extracellular protein is greater than the normal amount of cleaved extracellular protein, then the test compound is an agonist of granzyme B. Similar assays may be used to identify a rate of elastin cleavage by granzyme B in the presence or absence of a particular inhibitor, antagonist or agonist.

Skin is comprised of three main layers: the epidermis, the dermis and subcutaneous layers. Each of these three layers has individual compositions. The functions and structures of these layers are known to a person of skill in the art. The epidermis is the outermost layer of skin and includes both living and dead cell layers. The dermis is the middle layer of skin and is comprised of arrangements of collagen fibres, which surround many specialized cells and structures. Hair follicles are found within the dermis, and produce the hair shaft which grows out through layers of the dermis and epidermis to become visible as hair. The lowermost layer of the skin is the subcutaneous layer, often called the sub-dermis. The subcutaneous layer is comprised largely of fat and connective tissue and houses larger blood vessels and nerves. Elastin may be found in all layers of the skin, but is most prominently in the dermis layer.

A youthful appearance is achieved by not having at least one of the characteristic signs of age. This is often achieved by being young. Nevertheless, there are circumstances in which being young does not confer a youthful appearance as a disease or disorder or other non-time related event has conferred the characteristics associated with age. A youthful appearance is often characterized by the condition of the skin and the following skin qualities are typically associated with, but not limited to, a youthful appearance: small pore size, healthy skin tone, radiance, clarity, tautness, firmness, plumpness, suppleness, elasticity, softness, healthy skin texture, healthy skin contours, such as few or no wrinkles, shallow wrinkle depth, few or no fine lines, healthy skin luster and brightness, moisturized skin, healthy skin thickness and resilient skin. If a skin of a subject comprises any one or more of these characteristics then a youthful appearance is achieved.

The appearance of ageing can occur for a variety of reasons, but typically happens at a normal rate associated with the passage of time. A rate of appearance of ageing will be different for different subjects, depending on a variety of factors including age, gender, diet and lifestyle. An appearance of ageing is often characterized by the condition of the skin. Characteristics associated with an appearance of ageing in the skin include, but are not limited to, skin fragility, skin atrophy, skin wrinkles, fine lines, skin discolouration, skin sagging, skin fatigue, skin stress, skin inelasticity, skin fragility, skin softening, skin flakiness, skin dryness, enlarged pore size, skin thinning, reduced rate of skin cell turnover, deep and deepening of skin wrinkles. The rate of appearance of ageing can be measured by measuring the rate at which any one or more of the above characteristics appear. An appearance of ageing may be inhibited, reduced or treated by reducing or maintaining a state of any one or more of these skin characteristics.

In many circumstances a reduction in the appearance of ageing of skin occurs when, by inhibiting granzyme B, the rate of elastin formation is caused to exceed the rate of elastin cleavage. In many other circumstances, a youthful appearance of skin is maintained when, by inhibiting granzyme B, the rate of elastin formation is caused to equal the rate of elastin cleavage. In many other circumstances, a reduction in a rate of appearance of ageing of skin is achieved when, by applying a granzyme B inhibitor, the rate of elastin cleavage is slowed such that the rate of elastin cleavage exceeds the rate of elastin formation and the ratio of the rate of elastin cleavage to the rate of elastin formation is greater after application of the granzyme B inhibitor compared to the ratio before application of the granzyme B inhibitor. In many other circumstances, an extracellular protein, other than elastin, is also cleaved by granzyme B, and the beneficial effects of inhibiting granzyme B may be enhanced beyond what is realized by inhibiting elastin cleavage alone.

Many granzyme B inhibitors are known to a person of skill in the art and are, for example, described in international patent application published under WO 03/065987 and United States patent application published under US 2003/0148511 as well as in Bird et al. Mol. Cell. Biol. 18, 6387-6398 (1998) and Kam et al. Biochim Biophys Acta 1477(1-2):307:23 (2000). Granzyme B inhibitors include, but are not limited to, peptides and small molecules. Methods of identifying a granzyme B inhibitor are described elsewhere in this application.

Many granzyme B inhibitors are water soluble and may be formed as salts. In such cases, compositions of granzyme B inhibitors may comprise a physiologically acceptable salt, which are known to a person of skill in the art. Preparations will typically comprise one or more carriers acceptable for the mode of administration of the preparation, be it by topical administration, lavage, epidermal administration, sub-epidermal administration, dermal administration, sub-dermal administration, sub-cutaneous administration, systemic administration, injection, inhalation, oral, or other modes suitable for the selected treatment. Suitable carriers are those known in the art for use in such modes of administration.

Suitable compositions may be formulated by means known in the art and their mode of administration and dose determined by a person of skill in the art. For parenteral administration, compound may be dissolved in sterile water or saline or a pharmaceutically acceptable vehicle used for administration of non-water soluble compounds such as those used for vitamin K. For enteral administration, compound may be administered in a tablet, capsule, or dissolved in liquid form. The tablet or capsule may be enteric coated, or in a formulation for sustained release. Many suitable formulations are known including, polymeric or protein microparticles encapsulating a compound to be released, ointments, pastes, gels, hydrogels, foams, creams, powders, lotions, oils, semi-solids, soaps, medicated soaps, shampoos, medicated shampoos, sprays, films, or solutions which can be used topically or locally to administer a compound. A sustained release patch or implant may be employed to provide release over a prolonged period of time. Many techniques known to one of skill in the art are described in Remington: the Science & Practice of Pharmacy by Alfonso Gennaro, 20^{th} ed., Williams & Wilkins, (2000). Formulations may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of the compounds. Other potentially useful delivery systems for modulatory compounds include ethylene-vinyl acetate copolymer particles, osmotic pumps, implantable infusion systems, and liposomes. Formulations may contain excipients, for example, lactose, or may be aqueous solutions containing, for example, polyoxyethylene-9-to lauryl ether, glycocholate and deoxycholate, or may be oily solutions for administration in the form of drops, or as a gel.

Compositions containing granzyme B inhibitors may also include penetrating agents. Penetrating agents may improve the ability of the granzyme B inhibitors to be delivered to deeper layers of the skin. Penetrating agents that may be used are known to a person of skill in the art and include, but are not limited to, hyaluronic acid, insulin, liposome, or the like, as well as L-arginine or the arginine-containing amino acids.

Compounds or compositions of granzyme B inhibitors may be administered by means of a medical device or appliance such as an implant, graft, prosthesis, garment of clothing, stent, etc. Also, implants may be devised which are intended to contain and release such compounds or compositions. An example would be an implant made of a polymeric material adapted to release the compound over a period of time. Such implants may be placed into a garment to be worn by a subject, for example a glove, shirt, mask or hat.

An “effective amount” of a granzyme B composition includes a therapeutically effective amount or a prophylactically effective amount. A “therapeutically effective amount” refers to an amount effective, at dosages and for periods of time necessary, to achieve the desired therapeutic result, such as improved skin elasticity, skin durability, skin firming, and skin texture. A therapeutically effective amount of a compound may vary according to factors such as the skin state, age, sex, and weight of the subject, and the ability of the compound to elicit a desired response in the subject. Dosage regimens may be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as improved skin elasticity, skin durability, skin firming, and skin texture. Typically, a prophylactic dose is used in subjects prior to or at an earlier stage of skin deterioration, so that a prophylactically effective amount may be less than a therapeutically effective amount.

It is to be noted that dosage values may vary with the severity of the appearance of age of the skin. For any particular subject, specific dosage regimens may be adjusted over time according to the individual need and the judgement of the person applying or supervising the applying of the compositions. Dosage ranges set forth herein are exemplary only and do not limit the dosage ranges that may be selected. The amount of active compound(s) in the composition may vary according to factors such as the skin state, age, sex, and weight of the subject. Dosage regimens may be adjusted to provide the optimum therapeutic response. For example, a single application may be administered, several divided doses may be administered over time or the dose may be proportionally reduced or increased as indicated by the exigencies of the situation. It may be advantageous to formulate compositions in dosage unit form for ease of administration and uniformity of dosage.

In general, compounds of the invention should be used without causing substantial toxicity. Toxicity of the compounds of the invention can be determined using standard techniques, for example, by testing in cell cultures or experimental animals and determining the therapeutic index, i.e., the ratio between the LD50 (the dose lethal to 50% of the population) and the LD100 (the dose lethal to 100% of the population). In some circumstances however, such as in severe appearance of ageing of skin, it may be necessary to administer substantial excesses of the compositions.

As used herein, a “subject” may be a mammal, non-human primate, domestic pet, human, rat, mouse, cow, horse, pig, sheep, goat, dog, cat, etc. The subject may be suspected of having or at risk for having an appearance of ageing of the skin. Diagnostic methods for various stages of the appearance of ageing of skin, including skin wrinkling and skin sagging, are known to those of ordinary skill in the art, see for example, Measuring the Skin by Agache et al., Springer (2004).

Granzyme B inhibitors may be used to inhibit or reduce the appearance of ageing. Ageing is a natural phenomenon that cannot be reversed per se, but the appearance of ageing, such as skin deterioration including, but not limited to, skin inelasticity, skin fragility, skin softening, skin flakiness, skin dryness, enlarged pore size, skin thinning, reduced rate of skin cell turnover, skin wrinkling, deepening of skin wrinkles, skin sagging, fine lines, and skin discolouration may be inhibited or reduced.

Granzyme B inhibitors may be used to increase or decrease a rate of increasing or a rate of decreasing occurrences of a particular skin characteristic. In other words, a granzyme B inhibitor, when applied to the skin or a portion of the skin of a subject delays the onset of an appearance of aging. For example, in a population of subjects where half of the population applies a granzyme B inhibitor to their skin and another half of the population does not apply a granzyme B inhibitor to their skin, the half which applied a granzyme B inhibitor would not appear as aged as the half which did not apply the granzyme B inhibitor after a period of time had elapsed. The half of the population which applied a granzyme B inhibitor to the skin would also have maintained a youthful appearance.

The rate at which a particular subject experiences a change in the rate of appearance of a particular skin characteristic, i.e. an increasing or decreasing rate of the appearance of a particular skin characteristic will depend on a variety of factors, including, but not limited to age, weight, sex and lifestyle of the subject. As such, rates are not necessarily constant, but a normal rate of increasing or decreasing of an appearance of a characteristic, defined as being the new occurrence of a particular characteristic over a predetermined period of time under a set of conditions that do not include the presence of a granzyme B inhibitor applied by a method or use of this invention, is increased or decreased by applying a granzyme B inhibitor in accordance with a method or use of this invention. Methods of measuring skin characteristics, rates of increasing appearance of skin characteristics and rates of decreasing appearance of skin characteristics are known to a person of skill in the art, see for example, Measuring the Skin by Agache et al., Springer (2004).

Surprisingly, granzyme B inhibitors may also be used to increase the density of hair follicles of a skin of a subject and may be used to reduce the occurrences of cutaneous xanthomas of a skin of a subject. Actively growing hair follicles contain melanocytes that transfer pigment to matrix keratinocytes, imparting colour to hair. Additionally, sebum, produced in sebaceous glands, is often secreted via hair follicles. Increased density of hair follicles results in increased pigment production and increased sebum secretion resulting in improved hair appearance (e.g. hair that is less grey in colour or not grey at all) as well as healthier hair and skin. Granzyme B inhibitors also cause hair follicles to appear deeper in the skin which provide stronger hair that is less susceptible to mechanical damage. Additionally, a characteristic sign of ageing is the reduction in hair follicle density. It is known in the art that age and follicular miniaturization are weak predictors of total hair count (see Chapman et al., British Journal of Dermatology (2005), 152: 646-649). Consequently, the characteristic sign of age associated with hair follicle density is not predictive of hair density.

A granzyme B inhibitor or composition comprising a granzyme B inhibitor may be applied to a portion of the skin of a subject or to the whole of the skin of the subject. For example, granzyme B inhibitors and composition comprising granzyme B inhibitors may be applied to the skin, only on the face, only on the scalp, on the whole head or to each part of the body.

Various alternative embodiments and examples of the invention are described herein. These embodiments and examples are illustrative and should not be construed as limiting the scope of the invention.

### Example 1

### ApoE/Granzyme B Double Knock-out Mice

Four groups of mice consisting of (1) C57B1/6 wild-type (WT), (2) C57/ApoE −/− (ApoE-KO), (3) C57/GrB −/− (GrB-KO), and (4) C57 GrB/ApoE-DKO (DKO) were fed a normal chow or high fat ‘Western’ diet (21% fat, 0.2% cholesterol) for 30 weeks. No obvious phenotypic differences were observed in these mice during the first 3 months. Mice were sacrificed and tissues harvested at 30 weeks of age. In accordance with previous reports in the literature, the ApoE-KO mice had developed severe skin xanthomatosis, hair loss, hair discoloration and numerous atherosclerotic lesions.

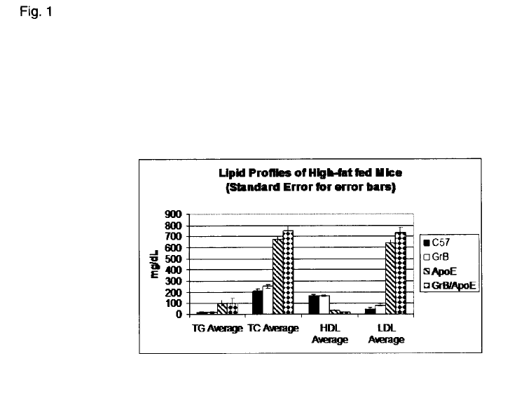

There is no difference between the ApoE-KO and the DKO mice with respect to blood cholesterol and lipoprotein levels. Total cholesterol and LDL-C plasma concentrations are the same in both groups of mice. No significant differences in HDL, LDL and triglycerides are observed between ApoE-KO (hatched bars) and DKO (checked bars) mice fed a Western diet (FIG. 1). Removal of granzyme B activity alone (white bars) does not have a significant effect on the blood lipid profiles compared to the C57/B16 (black bar).

At 30 weeks, the DKO mice have no visible xanthomas (Table 1). The DKO mice have smooth and unwrinkled skin.

TABLE 1
Animals affected with cutaneous xanthomas.

| Strain | # Animals with xanthomas/total |
|---|---|
| C57Bl/6 | 0/14 |
| GrB-KO | 0/17 |
| ApoE-KO | 32/32 |
| GrB/apoE-DKO | 0/13 |

Values indicate # animals with xanthomas/total # animals.

The fur in the ApoE-KO mice is patchy, discoloured (grey hair colour) and held weakly in the skin (easily removed by depilatory), while the DKO mice retain their dark fur and does not discolour or show grey hair colour, and is held firmly in the skin. The DKO mice's fur is held even more firmly than the GrB-KO mice. The hair follicles in the GrB-KO and the DKO mice are more abundant and embedded deeper in the fatty layer of the skin, compared to the wild-type or the ApoE-KO mice (Table 2). A standard Nair-mediated hair removal procedure takes more than 45 minutes in the GrB-KO and DKO mice, compared to 5 minutes in the WT or ApoE-KO mice.

TABLE 2
Hair follicle density of skin samples of mouse strains.

| | Epidermis and | Subcutaneous |
|---|---|---|
| Strain | Dermal Layer | Layer |
| C57Bl/6 | 15 | 11 |
| GrB-KO | 22 | 7 |
| ApoE-KO | 13 | 3 |
| GrB/apoE-DKO | 47 | 33 |

Values indicate # follicles per 8.9 mm².
N = 8 per strain.

### Example 2

### Elastin and Granzyme B Distribution

Colocalization of granzyme B and macrophages in the lesions of the aortic roots were performed and imaged by confocal microscopy. The lesion of the ApoE-KO mice showed both granzyme B and macrophage staining, however colocalization of both occurred at specific regions of the plaque: the fibrotic cap and the shoulder regions. Granzyme B staining was localized at the internal elastic lamina.

In order to adhere to the aortic walls, smooth muscle cells require elastin. Aortas of C57 wt, GrB-KO, ApoE-KO and DKO mice were stained with elastic van Gieson. The aortic wall of the ApoE mouse in the stained aortic section appeared very thin and elastin staining is markedly reduced compared to the stained aortic section of the C57 wt mouse. In the stained aortic section of the DKO mouse, the aorta wall appeared significantly thicker and elastin staining was correspondingly more intense. Granzyme B also colocalized with the internal elastic lamina of atherosclerotic plaques and an influx of macrophages in the ApoE-KO. This colocalization was not observed in the DKO mice by confocal microscopy staining.

### Example 3

### Reduced Cutaneous Inflammation and Improved Skin Appearance in DKO Mice

The appearance of the mice was observed and the skin of ApoE-KO mice appeared much more aged, unhealthy and was very fragile. The skin had markedly reduced elasticity. The DKO mice, where granzyme B activity was absent, did not exhibit this reduced elasticity. An area of massive immune cell infiltration in the ApoE-KO mice was visible, that was also not observed in the DKO mice.

The skin of the DKO mice appeared thicker, stronger, more elastic, healthier in colour, and healthier in texture (e.g. less wrinkles) when compared to the ApoE-KO mice.

### Example 4

### Granzyme B Binds to the Extracellular Matrix Protein Elastin

An in vitro granzyme B elastin binding assay was conducted in the following manner. Granzyme B at 50, 100 and 300 ng was incubated with 15 μg of human insoluble skin (Sk) and aortic (Ao) elastin (Elastin Products Company Owensville, Mo.) in PBS for three hours at room temperature. The samples were centrifuged at 1000×g at room temperature for three minutes and the insoluble elastin collected in the pellet. The supernatants, which contained unbound granzyme B, were denatured with SDS loading buffer and run on a 10% SDS-PAGE gel. Granzyme B was detected by Western blot. Each gel contained three lanes: a first lane related to a sample containing granzyme B in the absence of elastin; a second lane related to the samples containing granzyme B and human insoluble skin elastin; and a third lane related to the sample containing granzyme B and aortic elastin. The lane relating to the sample containing granzyme B in the absence of elastin showed a heavy band in the supernatant and a faint band in the pellet. The lanes relating to the samples containing granzyme B and skin elastin, and granzyme B and aortic elastin both showed heavy bands in the pellet, which bands were much heavier than the faint band seen in the pellet relating to the sample containing granzyme B in the absence of elastin. Furthermore, the band in the supernatant for the sample containing granzyme B and skin elastin was dramatically less pronounced than the supernatant band shown in the sample relating to granzyme B in the absence of elastin. No band appeared in the supernatant sample containing granzyme B and aortic elastin. Hence, there is less granzyme B present in the supernatant, thus indicating that granzyme B was associating with the elastin in the pellet. This phenomenon was dose-dependent and not restricted to the type of elastin used (i.e. skin elastin or aortic elastin).

### Example 5

### Granzyme B Cleaves Extracellular Matrix Proteins

Treatment of human coronary artery smooth muscle cells (SMC) matrix with granzyme B induced a cleavage of a number of extracellular proteins. Extracellular proteins from SMC cultures were biotinylated and incubated with granzyme B. The supernatant was collected at 2,4 and 24 hours after treatment, and the entire insoluble extracellular protein preparation collected at 24 hours. Extracellular proteins were visualized by Western blot for biotin. Western blot for beta-actin confirmed that the extracellular protein preparation was devoid of intercellular proteins. Western blots for fibronectin, phosphorylated FAK (p-FAK), and FAK were also performed on lysates of SMC treated with granzyme B. In the collected insoluble proteins, four protein bands between approximately 50-70 kDa and approximately 236 kDa disappeared 24 hours after treatment with granzyme B and cleavage of fragments approximately 25-39 kDa were evident in the matrix at this same time point. Further, the six proteins and/or cleavage fragments ranging in molecular weight from approximately 29-148 kDa were eluted into the supernatant as early as two hours after granzyme B treatment. To ensure that the SMC extracellular protein preparations used were devoid of intracellular proteins, western blotting for beta-actin was performed on the collected supernatant and extracellular proteins. Beta-actin was apparent in SMC lysates (positive control) but was absent from matrix and supernatant preparations.

To identify extracellular proteins that are cleaved by granzyme B, western blots for fibronectin, collagen, and vitronectin on lysates from untreated and granzyme B-treated SMCs were performed. In all SMCs treated with granzyme B for 24 hours, there was a reduction in the total amount of fibronectin in lysates collected from SMCs. In the supernatants of granzyme B-treated SMCs at 24 hours, a fibronectin cleavage product was detected. There was no cleavage of collagen IV or vitronectin was observed. Therefore, granzyme B induces a cleavage of fibronectin in SMC extracellular matrixes but does not affect collagen IV or vitronectin.

### Example 6

### Granzyme B Binds and Degrades Elastin In Vitro

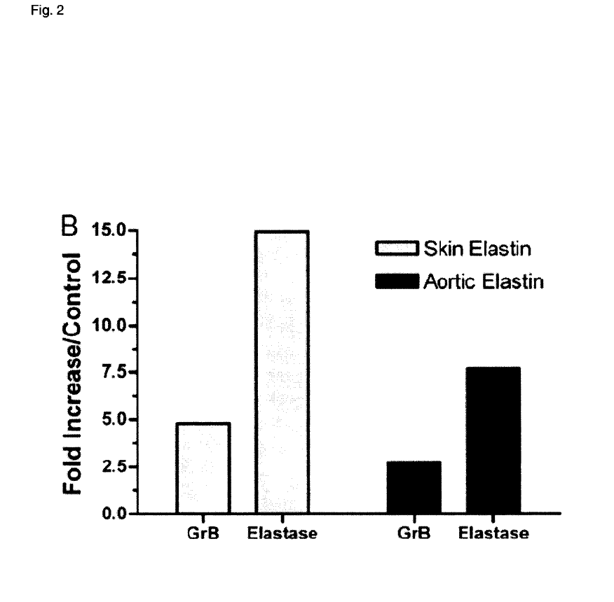

Tritiated elastin was prepared with the modifications as described in Banda, M. J. and Werb, Z. (1981) Biochem J 193: 589-605 and Gordon, S., Werb, Z. and Cohn, Z. A. (1976) in In Vitro Methods in Cell Mediated and Tumor Immunity, eds. Bloom, B. R. and David, J. R. (Academic Press, New York), pages 349-350. 1 mg of skin or aortic elastin was diluted in 1 ml dH₂O and pHed to 9.2. 1 mCi NaB₃H₄ (PerkinElmer, Waltham Mass.) and 2 mg of non-radioactive NaB₃H₄ (Sigma, St. Louis, Mo.) was added. After 2 hours of incubation, the pH was adjusted to 3.0 and the elastin was incubated for an additional 30 minutes. The elastin was centrifuged for 3 minutes at 5000×g and the pellet was repeatedly washed to remove excess NaB₃H₄. For the cleavage assays, 0.15 mg 3H-elastin was incubated with granzyme B (0.75 μg was added a total of 5 times) at room temperature for 7 days. At day 7 of incubation, 25 μg of elastase (Elastin Products Company, Owensville, Mo.) was incubated with elastin for 2 hours, as a positive control. After incubations, reactions were centrifuged at 5000×g for 3 minutes. The radioactivity of the soluble, cleaved elastin fragments in the supernatant was counted in Ready Safe Scintillation Fluid (Beckman-Coulter, Fullerton, Calif.). The radioactivity of the cleaved, soluble elastin fragments was 4.8 times and 2.7 times higher than background for skin and aortic elastin, respectively (FIG. 2). Proteolysis of elastin by elastase yielded a radioactivity increase over background of 14.9 fold for skin elastin and 7.7 fold for aortic elastin. These data show that granzyme B has affinity to elastin and has elastolytic activity.

Although the foregoing invention has been described in some detail by way of illustration and example for purposes of clarity of understanding, it will be readily apparent to those of skill in the art in light of the teachings of this invention that changes and modification may be made thereto without departing from the spirit or scope of the appended claims. All patents, patent applications and publications referred to herein are hereby incorporated by reference.

1. A method of maintaining a youthful appearance or reducing an appearance of ageing or inhibiting an appearance of ageing of a skin of a subject comprising applying a granzyme B inhibitor to the skin of the subject.

2-3. (canceled)

4. The method of claim 1 wherein the maintaining of a youthful appearance or the reducing of an appearance of ageing or the inhibiting of an appearance of ageing of the skin of a subject is determined by one or more of the following:
(a) extracellular protein content of the skin is maintained or increased;
(b) skin elastin content is maintained or increased;
(c) skin elasticity is maintained or increased;
(d) skin fragility is maintained or reduced;
(e) skin firmness is maintained or increased;
(f) skin flakiness is maintained or reduced;
(g) skin dryness is maintained or reduced;
(h) pore size of the skin is maintained or reduced;
(i) skin thickness is maintained or increased;
(j) rate of skin cell turnover is maintained or increased;
(k) appearance of wrinkles in the skin is maintained or reduced;
(l) depth of wrinkles is maintained or reduced;
(m) appearance of fine lines in the skin is maintained or reduced;
(n) appearance of skin discolouration is maintained or reduced;
(o) rate of decreasing extracellular protein content of the skin is reduced;
(p) rate of decreasing skin elastin content of the skin is reduced;
(q) rate of decreasing skin elasticity of the skin is reduced;
(r) rate of increasing skin fragility of the skin is reduced;
(s) rate of decreasing skin firmness of the skin is reduced;
(t) rate of increasing skin flakiness of the skin is reduced;
(u) rate of increasing skin dryness of the skin is reduced;
(v) rate of pore size enlargement of the skin is reduced;
(w) rate of decreasing skin thickness is reduced;
(x) rate of decreasing skin cell turnover is increased;
(y) rate of increasing appearance of wrinkles is reduced;
(z) rate of increasing depth of wrinkles is reduced;
(aa) rate of increasing appearance of fine lines is reduced; and
(bb) rate of increasing appearance of skin discolouration is reduced.

5-17. (canceled)

18. A method of reducing a rate of an appearance of ageing of a skin of a subject comprising applying a granzyme B inhibitor to the skin of the subject.

19. The method of claim 18 wherein the reducing a rate of an appearance of ageing of a skin of a subject is determined by one or more of the following:
(a) a rate of decreasing extracellular protein content of the skin is reduced;
(b) rate of decreasing skin elastin content of the skin is reduced;
(c) rate of decreasing skin elasticity of the skin is reduced;
(d) rate of increasing skin fragility of the skin is reduced;
(e) rate of decreasing skin firmness of the skin is reduced;
(f) rate of increasing skin flakiness of the skin is reduced;
(g) rate of increasing skin dryness of the skin is reduced;
(h) rate of pore size enlargement of the skin is reduced;
(i) rate of decreasing skin thickness is reduced;
(j) rate of decreasing skin cell turnover is increased;
(k) rate of increasing appearance of wrinkles is reduced;
(l) rate of increasing depth of wrinkles is reduced;
(m) rate of increasing appearance of fine lines is reduced; and
(n) rate of increasing appearance of skin discolouration is reduced.

20-32. (canceled)

33. A method of reducing a skin inelasticity or reducing a rate of increasing skin inelasticity or maintaining a skin elasticity in a subject comprising applying a granzyme B inhibitor to a skin of the subject.

34-35. (canceled)

36. A method of increasing the density of hair follicles in a subject comprising applying a granzyme B inhibitor to a skin of the subject.

37. The method of claim 36 wherein a grey hair colour is reduced.

38. The method of claim 1 wherein the granzyme B inhibitor is applied topically or sub-dermally.

39. (canceled)

40. The method of claim 38 wherein the granzyme B inhibitor is applied to all of the skin or to a portion of the skin or only to a scalp of the subject.

41-42. (canceled)

43. The method of claim 1 wherein the granzyme B inhibitor is administered systemically.

44. The method of claim 1 wherein the subject is a mammal.

45. The method of claim 44 wherein the subject is a domestic pet or a human.

46. (canceled)

47. The method of claim 44 wherein the subject is a dog.

48-149. (canceled)

150. A method of identifying a granzyme B inhibitor or agonist comprising:
i) contacting granzyme B with a test compound thereby forming a primed granzyme B;
ii) contacting the primed granzyme B with an extracellular skin membrane; and
iii) measuring the amount of cleaved extracellular protein,
wherein low levels of cleaved extracellular protein indicate the test compound is an inhibitor of granzyme B, and wherein high levels of cleaved extracellular protein indicate the test compound is an agonist of granzyme B.

151. The method of claim 150, wherein the cleaved extracellular protein measured is elastin.

152-155. (canceled)
